(19) United States
(12) Patent Application Publication
(10) Pub. No.: US 2003/0166663 A1
(43) Pub. Date: Sep. 4, 2003

(54) Use

(76) Inventor: Patrizia Caldirola, Uppsala (SE)

Correspondence Address: JEFFREY D. HSI, Fish & Richardson P.C., 225 Franklin Street, Boston, MA, 02110-2804, US

(21) Appl. No.: 10/290,915

(22) Filed: Nov. 8, 2002

Related U.S. Application Data
(60) Provisional application No. 60/356,890, filed on Feb. 13, 2002.

(30) Foreign Application Priority Data
Nov. 9, 2001 (SE) 0103767-0

(51) Int. Cl. A61K 31/496

(52) U.S. Cl. 514/253.06

(57) ABSTRACT

The invention provides methods of treatment or prophylaxis of obesity, or methods of treatment for the reduction of food intake, comprising administering to a patient in need of such treatment a therapeutically effective amount of a sulfonamide compound of Formula I:

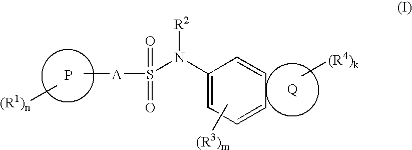

wherein the substituents are as described in the specification.

### CROSS-REFERENCE TO RELATED APPLICATIONS

This application claims benefit of Swedish application No. 0103767-0, filed Nov. 9, 2001 and U.S. provisional application Ser. No. 60/356,890, filed Feb. 13, 2002, each of which is incorporated by reference in its entirety.

### TECHNICAL FIELD

The present invention relates to the use of sulfonamides and their derivatives, which bind selectively to 5-HT₆ receptors, in the treatment of obesity or for the reduction of food intake.

### BACKGROUND ART

Obesity is a condition characterized in an increase in body fat content resulting in excess body weight above accepted norms. Obesity is the most important nutritional disorder in the western world and represents a major health problem in all industrialized countries. This disorder leads to increased mortality due to increased incidences of diseases such as cardiovascular disease, digestive disease, respiratory disease, cancer and NIDDM (type II diabetes). Searching for compounds that reduce body weight has been going on for many decades. One line of research has been activation of serotonergic systems, either by direct activation of serotonin receptor subtypes or by inhibiting serotonin reuptake. The exact receptor subtype profile required is however not known.

Serotonin (5-hydroxytryptamine or 5-HT), a key transmitter of the peripheral and central nervous system, modulates a wide range of physiological and pathological functions, including anxiety, sleep regulation, aggression, feeding and depression. Multiple serotonin receptor subtypes have been identified and cloned. One of these, the 5-HT₆ receptor, was cloned by several groups in 1993 (Ruat et al. (1993) Biochem. Biophys. Res. Commun., 193: 268-276; Sebben et al. (1994) NeuroReport 5: 2553-2557) This receptor is positively coupled to adenylyl cyclase and displays affinity for antidepressants such as clozapine. Recently, the effect of 5-HT₆ antagonist and 5-HT₆ antisense oligonucleotides to reduce food intake in rats has been reported (Bentley et al. (1999) Br. J. Pharmac. Suppl. 126: P66; Bentley et al. (1997) J. Psychopharmacol. Suppl. A64: 255).

WO01/32646 discloses sulfonamide compounds as ligands selective for the 5-HT₆ receptors, and of proposed value in the treatment or prevention of CNS disorders, including Alzheimer's disease, Parkinson's disease, schizophrenia, depression and anxiety. However, it has not been disclosed that such derivatives are useful for the treatment of obesity.

### BRIEF DESCRIPTION OF THE DRAWINGS

FIG. 1 is a graph depicting the effect on food intake in obese mice by administration of 5-chloro-3-methyl-benzo[b]thiophene-2-sulphonic acid (4-[4-methylpiperazin-1-yl] quinolin-6-yl)amide.

FIG. 2 is a graph depicting the effect on food intake in obese mice by administration of 4-n-butyl-N-(4-piperazin-1-yl-quinolin-6-yl)benzenesulphonamide.

### DISCLOSURE OF THE INVENTION

It has surprisingly been found that 5-HT₆ receptor antagonists, belonging to the class of sulfonamide compounds disclosed in WO01/32646 reduce food intake and body weight. Consequently, the present invention provides methods for the treatment or prophylaxis of obesity or for the reduction of food intake in mammals, including humans. The methods comprise administering to a patient in need of such treatment (e.g., identified as in need) a therapeutically effective amount of one or more compounds of formula (I) or a pharmaceutically acceptable salt or prodrug thereof:

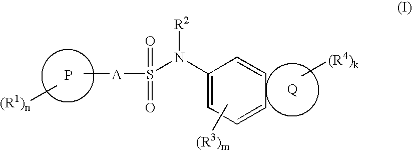

wherein

P is phenyl, naphthyl, a 5 or 6 membered heteroaryl ring containing 1 to 3 heteroatoms selected from oxygen, nitrogen and sulfur, or a bicyclic or tricyclic heteroaryl ring containing 1 to 3 heteroatoms selected from oxygen, nitrogen and sulfur;

A is a single bond, a C₁₋₆alkylene or a C₂₋₆alkenylene group;

Each R¹ is independently halogen, C₁₋₆alkyl optionally substituted by one or more halogen atoms, C₃₋₆-cycloalkyl, phenyl, COC₁₋₆alkyl, C₁₋₆alkoxy, OCF₃, hydroxy, hydroxy-C₁₋₆alkyl, hydroxy-C₁₋₆alkoxy, C₁₋₆alkoxy-C₁₋₆alkoxy, nitro, amino, C₁₋₆alkylamino, or di-C₁₋₆alkylamino;

n is 0, 1, 2, 3, 4 or5;

R² is hydrogen, C₁₋₆alkyl or together with a group R³ forms a group —(CR⁶R⁷)ₚ— where R⁶ and R⁷ are independently hydrogen or C₁₋₆alkyl and p is 2, 3 or 4;

Each R³ is independently C₁₋₆alkyl optionally substituted by one or more halogen atoms, halogen, C₁₋₆ alkoxy, or together with the group R² forms a group —(CR⁶R⁷)ₚ13 as defined above;

m is 0, 1 or 2;

Each R⁴ is independently C₁₋₆ alkyl or a group —X—R⁵ where X is a single bond, CH₂, O, NH or N—C₁₋₆alkyl;

kis 1 or 2; and

R⁵ is an optionally substituted 5- to 7-membered heterocyclic ring or a bicyclic heterocyclic ring containing 1 to 3 heteroatoms selected from nitrogen, sulfur or oxygen;

Q is a phenyl ring or is a 6-membered heteroaryl ring containing one or two nitrogen atoms.

In some embodiments of the invention, the body weight disorder to be addressed is obesity in a human, defined as a condition where the individual has a Body Mass Index (“BMI”), sometimes called Quetelet's Index, above currently accepted standards. BMI is calculated by dividing weight (in kg) by height² (in meters²). The current standards for both men and women accepted as “normal” are a BMI of 20-24.9 kg/m². Grade I obesity corresponds to a BMI of 25-29.9 kg/m²; Grade II obesity corresponds to a BMI of 30-40 kg/M² ; and Grade III obesity corresponds to a BMI greater than 40 kg/m². (E. Jequier, “Energy, obesity, and body weight standards,” Am. J Clin. Nutr., 45:1035-47 (1987)). Ideal body weight will vary among species and individuals based on height, body build, bone structure, and sex.

The obesity herein may be due to any cause, whether genetic or environmental. Examples of causes that may result in obesity or be the cause of obesity include overeating, diet rich in fats or sugars, environmental causes, medications, pathological conditions showing reduced metabolic activity or a decrease in resting energy expenditure as a percentage of total fat-free mass. Such induction of body weight increase is particularly suited for treatment using the compounds and compositions resulting from the use or process of the invention, or alternatively the methods of the invention. In this context, “treatment” of a body weight disorder refers e.g., to a reduction of an abnormally or pathologically elevated body weight, or, to a reduction of an abnormally high rate of increase in body weight.

Thus, in one aspect, the invention relates to a method for the inhibition and/or complete suppression of lipogenesis in obese mammals, i.e., the excessive accumulation of lipids in fat cells, which is one of the major features of human and animal obesity, or loss of total body weight, by administration of a compound of any of the formulae herein or a composition including a compound of any of the formulae herein.

Another aspect of the invention is a method for ameliorating the conditions that are a consequence of disease, such as preventing or arresting the progression of polycystic ovarian disease, so that the patient is no longer infertile, and increasing the insulin sensitivity and/or decreasing or eliminating the need or usage of insulin in a diabetic patient, e.g., one with adult-onset diabetes or Type II diabetes, by administration of a compound of any of the formulae herein or a composition including a compound of any of the formulae herein.

Still another aspect of the invention is a method for reducing the food intake in mammals, including humans, by administration of a compound of any of the formulae herein or a composition including a compound of any of the formulae herein.

The methods delineated herein can also include the step of identifying that the patient is in need of treatment for the aforementioned disorders or condition. The identification can be in the judgment of a patient or health care professional and can be subjective (e.g., opinion) or objective (e.g., measurable by a test or diagnostic method).

“Treatment” (of obesity) refers to reducing the BMI of the mammal to less than about 25.9, and maintaining that weight for at least 6 months. The treatment suitably results in a reduction in food or calorie intake by the mammal.

“Prevention” (of obesity) refers to preventing obesity from occurring if the treatment is administered prior to the onset of the obese condition. Moreover, if treatment is commenced in already obese subjects, such treatment is expected to prevent, or to prevent the progression of, the medical sequelae of obesity, such as, e.g., arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The term “halogen” is used herein to describe, unless otherwise stated, a group selected from fluorine, chlorine, bromine or iodine.

The expression “C₁₋₆alkyl” includes methyl and ethyl groups, and straight-chained, branched or cyclic propyl, butyl, pentyl and hexyl groups. Particular alkyl groups are methyl, ethyl, n-propyl, isopropyl and tert-butyl. Derived expressions such as “C₁₋₆alkoxy” and “C₁₋₆alkylamino” are to be construed accordingly.

The expression “C₁₋₆alkylene” as used herein refers to straight-chained and branched alkylene groups containing from 1 to 6 carbon atoms. Typical examples include methylene, ethylene, propylene and butylene groups.

The expression “C₂₋₆ alkenylene” as used herein refers to straight-chained and branched alkenylene groups containing from 2 to 6 carbon atoms. Typical examples include vinylene, allyl, dimethylallyl and butenylene groups.

The term “heteroaryl” refers to an aromatic 5-8 membered monocyclic, 8-12 membered bicyclic, or 11-14 membered tricyclic ring system having carbon atoms and 1-3 heteroatoms if monocyclic, 1-6 heteroatoms if bicyclic, or 1-9 heteroatoms if tricyclic, said heteroatoms selected from O, N, or S, wherein 0, 1, 2, 3, or 4 atoms of each ring may be substituted by a substituent.

The term “heterocyclic” refers to a nonaromatic 5-8 membered monocyclic, 8-12 membered bicyclic, or 11-14 membered tricyclic ring system having carbon atoms and 1-3 heteroatoms if monocyclic, 1-6 heteroatoms if bicyclic, or 1-9 heteroatoms if tricyclic, said heteroatoms selected from O, N, or S, wherein 0, 1, 2 or 3 atoms of each ring may be substituted by a substituent. When P is naphthyl this is intended to denote both 1-naphthyl and 2-naphthyl groups. When P is a 5 or 6-membered heteroaryl ring suitable examples include thienyl, furyl, pyrrolyl, triazolyl, diazolyl, oxazolyl, thiazolyl, oxadiazolyl, isothiazolyl, isoxazolyl, thiadiazolyl, pyridyl, pyrimidyl and pyrazinyl. When P is a bicyclic heteroaryl ring suitable examples include indolyl, benzofuryl, benzothienyl, quinolinyl and isoquinolinyl. When P is a tricyclic heteroaryl ring a preferred example is dibenzofuryl. The heteroaryl rings can be linked to the remainder of the molecule via any suitable carbon atom or, when present, a nitrogen atom.

Preferably P is phenyl, naphthyl, benzofuryl or benzothienyl.

Preferably A is a single bond, a methylene or ethylene group or a —CH═CH group. Most preferably A is a single bond.

When n is more than 1 the groups R¹ can be the same or different. Preferably R¹ is halogen (particularly chloro or bromo), or a C₁₋₆alkyl group optionally substituted by one or more halogen atoms, for example, methyl, ethyl, isopropyl, t-butyl or trifluoromethyl. Preferably n is 0, 1, 2 or 3, particularly 1 or 2.

When R² together with a group R³ forms a further group —(CR⁶R⁷)ₚ— both of the groups R⁶ and R⁷ are preferably hydrogen and p is preferably 2.

R² is preferably hydrogen.

A substituent R³ can be attached at any unsubstituted carbon atom within the fused ring. When m is more than 1 the groups R³ can be the same or different. It will be appreciated that when the R²/R³ groups are linked together, the group R³ must be attached to one of the carbon atoms of the fused ring with an ortho relationship with respect to the sulfonamide linkage.

Preferably m is 0.

The group R⁴ can be attached at any unsubstituted carbon atom within the ring Q. When k is more than 1 the groups R⁴ can be the same or different.

When R⁵ is a 5- to 7-membered heterocyclic ring suitable examples include piperazinyl, piperidyl, pyrrolidinyl and morpholinyl. The 5- to 7-membered heterocyclic rings can be linked to the remainder of the molecule via a carbon atom or, when present, a suitable nitrogen atom. It will be appreciated however, that when X is O, NH or N—C₁₋₆alkyl then the 5- to 7-membered heterocyclic ring must be linked to the rest of the molecule via a carbon atom. Preferably X is a single bond (i.e. R⁴=R⁵) and the 5- to 7-membered heterocyclic ring is attached to the rest of the molecule via a suitable nitrogen atom.

When R⁵ is a bicyclic heterocyclic ring, X is preferably a single bond (i.e. R⁴=R⁵) and suitable examples of such groups are

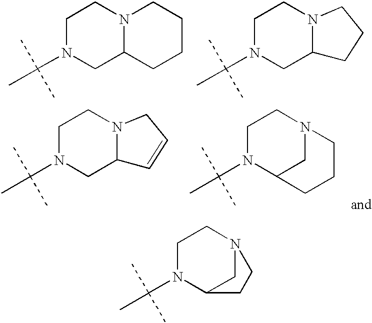

Optional substituents for each atom in the rings within the definition of R⁵, which can be present on carbon and/or nitrogen atoms, include C₁₋₆alkyl, in particular methyl.

Most preferably R⁴ is an unsubstituted piperazine or N-methyl piperazine attached to the rest of the molecule via a suitable nitrogen atom.

Suitably Q is a phenyl ring or is a 6 membered heteroaryl ring containing one or two nitrogen atoms. Preferably Q, together with the phenyl ring to which it is fused, forms a quinoline, isoquinoline or quinazoline ring.

Most preferably Q, together with the phenyl ring to which it is fused, forms a quinoline ring, and the substituent R⁴ is at the 4-position, that is to say, a group of formula (A)

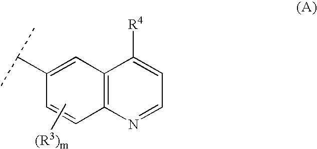

Particular preferred compounds of this invention include:

5-chloro-3-methyl-benzo[b]thiophene-2-sulfonic acid (4-[4-methylpiperazin-1-yl]quinolin-6-yl)amide,

5-chloro-naphthalene-2-sulfonic acid (4-[4-methyl-piperazin-1-yl]-quinolin-6-yl)amide,

4-bromo-N-[4-(4-methyl-piperazin-1-yl)-quinolin-6-yl]benzenesulfonamide,

3,5-dichloro-N-[4-(4-methyl-piperazin-1-yl)-quinolin-6-yl]benzenesulfonamide,

5-chloro-3-methyl-benzo[b]thiophene-2-sulfonic acid [4-(3,5-dimethylpiperazin-1-yl)-quinolin-6-yl]amide,

5-chloro-3-methyl-benzo[b]thiophene-2-sulfonic acid [4-(4-methyl-piperazin-1-yl)-quinazolin-6-yl]amide,

5-chloro-3-methyl-benzo[b]thiophene-2-sulfonic acid (4-piperazin-1-yl-quinolin-6-yl) amide,

3,5-dichloro-N-(4-piperazin-1-yl-quinolin-6-yl)benzenesulfonamide,

5-chloro-3-methyl-benzofuran-2-sulfonic acid (4-piperazin-1-yl-quinolin-6-yl)amide,

5,7-dichloro-3-methyl-benzo[b]thiophene-2-sulfonic acid (4-piperazin-1-yl-quinolin-6-yl)amide,

5-chloro-naphthalene-2-sulfonic acid (4-piperazin-1-yl-quinolin-6-yl)amide,

5-chloro-naphthalene-1-sulfonic acid (4-piperazin-1-yl-quinolin-6-yl)amide,

5-chloro-2-methyl-benzo[b]thiophene-3-sulfonic acid (4-piperazin-1-yl-quinolin-6-yl)amide,

2-dibenzofuran-sulfonic acid (4-piperazin-1-yl-quinolin-6-yl)amide,

5-chloro-3,7-dimethyl-benzo[b]thiophene-2-sulfonic acid (4-piperazin-1-yl-quinolin-6-yl)amide,

7-chloro-2-methyl-benzo[b]thiophene-3-sulfonic acid (4-piperazin-1-yl-quinolin-6-yl)amide,

4,6-dichloro-2-methyl-benzo[b]thiophene-3-sulfonic acid (4-piperazin-1-yl-quinolin-6-yl)amide,

5,7-dichloro-2-methyl-benzo[b]thiophene-3-sulfonic acid (4-piperazin-1-yl-quinolin-6-yl)amide,

biphenyl-4-sulfonic acid (4-piperazin-1-yl-quinolin-6-yl)amide,

4-tert-butyl-N-(4-piperazin-1-yl-quinolin-6-yl)benzenesulfonamide,

5-bromo-3-methyl-benzo[b]thiophene-2-sulfonic acid (4-piperazin-1-yl-quinolin-6-yl)amide,

4-n-butyl-N-(4-piperazin-1-yl-quinolin-6-yl)benzenesulfonamide,

4-chloro-2,5-dimethyl-N-(4-piperazin-1-yl-quinolin-6-yl)benzenesulfonamide,

5-chloro-3-ethyl-benzo[b]thiophene-2-sulfonic acid (4-piperazin-1-yl-quinolin-6-yl)amide,

5-chloro-3-isopropyl-benzo[b]thiophene-2-sulfonic acid (4-piperazin-1-yl-quinolin-6-yl)amide,

4-iodo-N-(4-piperazin-1-yl-quinolin-6-yl)benzenesulfonamide,

1-(5-chloro-3-methyl-benzo[b]thiophene-2-sulfonyl)-8-(4-methyl-piperazin-1-yl)-2,3-dihydro-1H-pyrrolo[2,3-g]quinoline,

5-chloro-naphthalene-2-sulfonic acid (2-methyl-4-piperazin-1-yl-quinolin-6-yl)amide,

5-chloro-3-methyl-benzo[b]thiophene-2-sulfonic acid (2-methyl-4-piperazin-1-yl-quinolin-6-yl)amide,

5-chloro-3-methyl-benzofuran-2-sulfonic acid (2-methyl-4-piperazin-1-yl-quinolin-6-yl)amide,

5-chloro-naphthalene-2-sulfonic acid (3-methyl-4-piperazin-1-yl-quinolin-6-yl)amide,

5-chloro-3-methyl-benzo[b]thiophen-2-sulfonic acid (3-methyl-4-piperazin-1-yl-quinolin-6-yl)amide,

5,7-dichloro-3-methyl-benzo[b]thiophen-2-sulfonic acid (3-methyl-4-piperazin-1-yl-quinolin-6-yl)amide,

5-chloro-3-methyl-benzofuran-2-sulfonic acid (3-methyl-4-piperazin-1-yl-quinolin-6-yl)amide,

4-tert-butyl-N-[4-(s-hexahydro-pyrrolo[1,2-a]pyrazin-2-yl)-quinolin-6-yl-benzenesulfonamide,

5-chloro-3-methyl-benzo[b]thiophen-2-sulfonic acid [4-(S-hexahydro-pyrrolo[1,2a]pyrazin-2-yl)-quinolin-6-yl]amide,

5-chloro-2-methyl-benzo[b]thlophene-3-sulfonic acid (4-[3,5-dimethylpiperazin-1-yl]quinolin-6-yl)amide,

5-chloro-3-methyl-benzo[b]thiophene-2-sulfonic acid [4-((S)-3-methyl-piperazin-1-yl)-quinotin-6-yl]amide,

5-chloro-3-methyl-benzo[b]thiophene-2-sulfonic acid [4-((R)-3-methyl-piperazin-1-yl)-quinolin-6-yl]amide,

5-chloro-3-methyl-benzo[b]thiophene-2-sulfonic acid [4-((R)-3-isopropyl-piperazine-1-yl)-quinolin-6-yl]amide,

5-chloro-3-methyl-benzo[b]thiophene-2-sulfonic acid [4-(trans-2,5-dimethyl-piperazine-1-yl)-quinolin-6-yl]amide,

5-chloro-3-methyl-benzo[b]thiophene-2-sulfonic acid [8-(4-methyl-piperazin-1-yl) naphthalen-2-yl]amide

or a pharmaceutically acceptable salt thereof.

As used herein, the term “body weight disorders” refers to the disorders caused by an imbalance between energy intake and energy expenditure, resulting in abnormal body weights. Such body weight disorders include obesity, and in particular acquired obesity. The “acquired obesity” includes diet-induced obesity that is caused by a certain diet, such as a high-fat diet, or a high-calorie diet.

Examples of disorders that may result in obesity or be the cause of obesity include overeating and bulimia, polycystic ovarian disease, craniopharyngioma, the Prader-Willi syndrome, Frochlich's syndrome, Type II diabetes, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia. The methods herein are useful for treating or preventing these disorders.

For use in medicine, the salts of the compounds of Formula I will be pharmaceutically acceptable salts. Other salts may, however, be useful in the preparation of the compounds of Formula I or of their pharmaceutically acceptable salts. Suitable pharmaceutically acceptable salts of the compounds of Formula I include acid addition salts which may, for example, be formed by mixing a solution of the compound according to the invention with a solution of a pharmaceutically acceptable acid such as for example maleic, hydrochloric, hydrobromic, phosphoric, acetic, fumaric, salicylic, citric, lactic, mandelic, tartaric and methanesulfonic.

Compounds of Formula (I) may also form solvates such as hydrates, and the invention also extends to these forms. When referred to herein, it is understood that the term ‘compound of Formula (I)’ also includes these forms.

Certain compounds of Formula (I) are capable of existing in stereoisomeric forms including diastereomers and enantiomers and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

“An effective amount” refers to an amount of a compound that confers a therapeutic effect on the treated subject. The therapeutic effect may be objective (i.e., measurable by some test or marker) or subjective (i.e., subject gives an indication of or feels an effect). The dose level of the compounds of formula I, and the frequency of dosage of the specific combination, will vary depending on a variety of factors including the potency of each specific compound employed, the metabolic stability and length of action of that compound, the patient's age, body weight, general health, sex, diet, mode and time of administration, rate of excretion, drug combination, the severity of the condition to be treated, and the patient undergoing therapy.

The daily dosage may, for example, range from about 0.001 mg to about 150 mg, preferably from about 0.01 mg to about 100 mg especially from about 0.1 to about 50 mg of the compound of formula I, administered singly or multiply in doses, e.g. dosages of from about 0.01 mg to about 25 mg each. Usually, such a combined dosage is given orally, but e.g. parenteral or rectal administration may also be chosen. A currently preferred oral daily dosage for a human subject is from about 1 to about 80 mg, preferably from about 1 to about 50 mg per day.

The present invention includes within its scope the use of prodrugs of the compounds of Formula I above. In general, such prodrugs will be functional derivatives of the compounds of Formula I which are readily convertible in vivo into the required compounds of Formula I. Conventional procedures for the selection and preparation of suitable prodrug derivatives are described, for example, in Design of Prodrugs, ed. H. Bundgaard, Elsevier, 1985.

Combinations of substituents and variables envisioned by this invention are only those that result in the formation of stable compounds. The term “stable”, as used herein, refers to compounds which possess stability sufficient to allow manufacture or shelf-stability, which maintains the integrity of the compound for a sufficient period of time to be useful, including for the purposes detailed herein (e.g., therapeutic administration to a subject for the treatment of obesity).

The compounds of Formula I, to be used according to the invention, can be prepared according to the methods described in WO01/32646.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

The invention will now be illustrated with the following examples, which however, are for illustrative purposes are not intended to limit the scope of the invention.

### EXAMPLES

### Effect of Compounds on Food Intake in ob/ob Mice

Animals

Obese (ob/ob) mouse is selected as the primary animal model for screening as this mutant mouse consumes high amounts of food resulting in a high signal to noise ratio. To further substantiate and compare efficacy data, the effect of the compounds on food consumption is also studied in wild type (C57BL/6J) mice. The amount of food consumed during 15 hours of infusion of compounds is recorded.

Male mice (obese C57BL/6JBom-Lep^{ob} and lean wild-type C57B1/6JBom; Bomholtsgaard, Denmark) 8-9 weeks with an average body weight of 50 g (obese) and 25 g (lean) are used in all the studies. The animals are housed singly in cages at 23±1° C., 40-60 % humidity and have free access to water and standard laboratory chow. The 12/12-h light/dark cycle is set to lights off at 5 p.m. The animals are conditioned for at least one week before start of study.

Compounds

The test compounds are dissolved in solvents suitable for each specific compound such as cyclodextrin, cyclodextrin/methane sulfonic acid, polyethylene glycol/methane sulfonic acid, or saline. Fresh solutions are made for each study. Doses of 30, 50 and 100 mg kg⁻¹ day⁻¹ are used. The purity of the test compounds is of analytical grade.

Minipump implantation

The animals are weighed at the start of the study and randomized based on body weight. Alzet osmotic minipumps (Model 2001D; infusion rate 8 μl/h) are used and loaded essentially as recommended by the Alzet technical information manual (Alza Scientific Products, 1997; Teeuwes and Yam, 1976). Continuous subcutaneous infusion with 24 hours duration is used. The minipumps are either filled with different concentrations of test compounds dissolved in vehicle or with only vehicle solution and maintained in vehicle pre-warmed to 37° C. (approx. 1 h). The minipumps are implanted subcutaneously in the neck/back region under short acting anesthesia (metofane/enflurane). This surgical procedure lasts approximately 5 min. It takes about 3 h to reach steady state delivery of the compound.

Food intake measurements

The weights of the food pellets are measured at 5 p.m. and at 8 p.m. for two days before (baseline) and one day after the implantation of the osmotic minipumps. The weighing is performed with a computer assisted Mettler Toledo PR 5002 balance. Occasional spillage is corrected for. At the end of the study the animals are killed by neck dislocation and trunk blood sampled for later analysis of plasma drug concentrations.

Determination of plasma concentration

The plasma sample proteins are precipitated with methanol, centrifuged, and the supernatant is transferred to HPLC vials and injected into the liquid chromatography/mass spectrometric system. The mass spectrometer is set for electrospray positive ion mode and Multiple Reaction Monitoring.

A linear regression analysis of the standards forced through the origin is used to calculate the concentrations of the unknown samples.

Statistical evaluation

Food consumption for 15 hours is measured for the three consecutive days and the percentage of basal level values is derived for each animal from the day before and after treatment. The values are expressed as mean±SD and mean±SEM from eight animals per dose group. Statistical evaluation is performed by Kruskal-Wallis one-way ANOVA using the per cent basal values. If statistical significance is reached at the level of p<0.05, Mann-Whitney U-test for statistical comparison between control and treatment groups is performed.

Formulation

5-Chloro-3-methyl-benzo[b]thiophene-2-sulfonic acid (4-[4-methylpiperazin-1-yl]quinolin-6-yl)amide was weighted in and dissolved to half of its final volume with a stock solution of PEG400 and 1.0% Tween 80. 100 mM Sodium Acetate was added to a final concentration of 10 mM. Purified water was added almost to final volume. The pH of the solution was measured and adjusted with 1M HCl. Qs to calculated weight with purified water. The solution was filtered through a 0.45 μm syringe filter (Millex HV).

Composition:

| | | | | | | |
|---|---|---|---|---|---|---|
| Example 1 | 2.3 | mg/ml | 6.9 | mg/ml | 23.0 | mg/ml |
| PEG 400 | 50% | w/v | 50% | w/v | 50% | w/v |
| Tween 80 | 0.5% | w/v | 0.5% | w/v | 0.5% | w/v |
| Sodium Acetate | 10 | mM | 10 | mM | 10 | mM |
| Properties: | | | | | | |

| | | | |
|---|---|---|---|
| pH | 5.3 | 5.2 | 5.2 |

### Example 1

### Effect on Food Intake of 5-chloro-3-methyl-benzo[b]thiophene-2-sulphonic acid (4-[4-methylpiperazin-1-yl]quinolin-6-yl)amide

TABLE 1

| | | | | | | % of basal | | | |
|---|---|---|---|---|---|---|---|---|---|
| | mg/kg/day | | Css, tot | | | level | | P vs. | Inhibition (%) |
| Compound | Nominal | Corrected | μM | SEM | n | Mean | SEM | Control | Based on basal |
| Vehicle | 0 | | | | 8 | 65.7 | 2.8 | | |
| EXAMPLE 1 | 10 | 10.4 | 1.00 | 0.04 | 8 | 62.5 | 6.4 | P < 0.7 | 4.9 |
| EXAMPLE 1 | 30 | 32.2 | 1.62 | 0.13 | 6 | 49.3 | 8.0 | P < 0.04 | 25.0 |
| EXAMPLE 1 | 100 | 99.1 | 1.81 | 0.15 | 7 | 49.9 | 6.7 | P < 0.02 | 24.1 |
| mCCP | 10 | 10.9 | 0.61 | 0.04 | 8 | 31.2 | 4.7 | P < 0.003 | 52.5 |

mCPP: m-chlorophenylpiperazine (reference compound)
Css, tot: total plasma exposure of the test compound and mCPP respectively at steady state

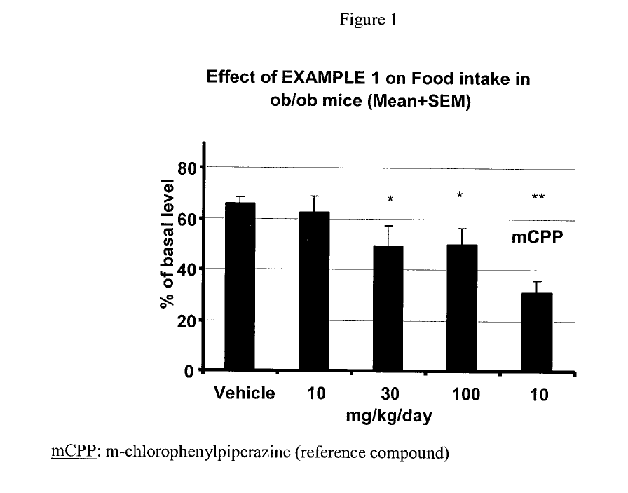

5-Chloro-3-methyl-benzo[b]thiophene-2-sulfonic acid (4-[4-methylpiperazin-1-yl]quinolin-6-yl)amide reduces food intake in ob/ob mice by 25% and 24% at 30 and 100 mg/kg/day respectively, as shown in Table 1. The effect of 5-Chloro-3-methyl-benzo[b]thiophene-2-sulfonic acid (4-[4-methylpiperazin-1-yl]quinolin-6-yl)amide on food intake as shown in % of basal level is provided in FIG. 1.

### Example 2

### Effect on Food Intake of 4-n-butyl-N-(4-piperazin-1-yl-quinolin-6-yl)benzenesulphonamide

TABLE 2

| | | | | | | % of basal | | | |
|---|---|---|---|---|---|---|---|---|---|
| | mg/kg/day | | Css, tot | | | level | | P vs. | Inhibition (%) |
| Compound | Nominal | Corrected | μM | SEM | n | Mean | SEM | Control | Based on basal |
| Vehicle | 1 | | | | 8 | 65.2 | 4.9 | | |
| EXAMPLE 2 | 10 | 10.9 | 0.37 | 0.05 | 8 | 61.0 | 3.0 | P < 0.40 | 6.5 |
| EXAMPLE 2 | 30 | 36.1 | 0.94 | 0.05 | 8 | 45.5 | 4.2 | P < 0.004 | 30.2 |
| EXAMPLE 2 | 100 | 117.0 | 1.29 | 0.09 | 8 | 43.6 | 5.1 | P < 0.002 | 33.2 |
| mCCP | 10 | 11.3 | 0.78 | 0.03 | 8 | 39.4 | 2.9 | P < 0.002 | 39.6 |

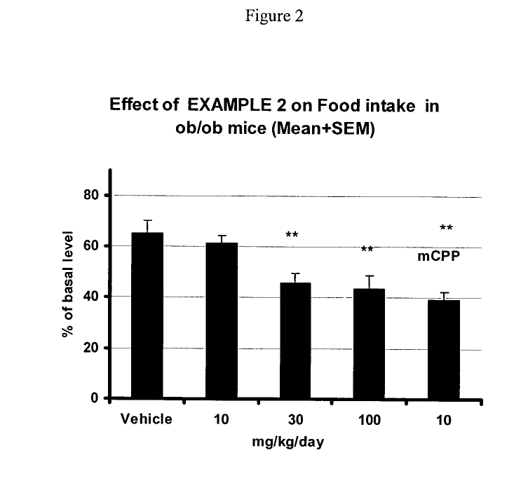

4-n-Butyl-N-(4-piperazin-1-yl-quinolin-6-yl)benzenesulfonamide reduces food intake in ob/ob mice by 30% and 33% at 30 and 100 mg/kg/day respectively, as shown in Table 2. The effect of 4-n-Butyl-N-(4-piperazin-1-yl-quinolin-6-yl)benzenesulfonamide on food intake as shown in % of basal level is provided in FIG. 2.

What is claimed is:

1. A method for the treatment and/or prevention of obesity, comprising administering to a patient in need of such treatment an effective amount of a compound, or a pharmaceutically acceptable salt or prodrug thereof, having a structure in accordance with Formula I: 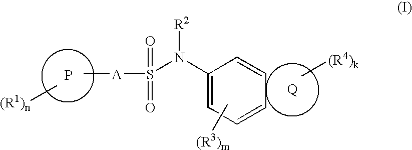
wherein
P is phenyl, naphthyl, a 5 or 6 membered heteroaryl ring comprising 1 to 3 heteroatoms selected from oxygen, nitrogen and sulfur, or a bicyclic or tricyclic heteroaryl ring comprising 1 to 3 heteroatoms selected from oxygen, nitrogen and sulfur;
A is a single bond, a C₁₋₆alkylene or a C₂₋₆alkenylene group;
each R¹ is independently halogen, C₁₋₆alkyl optionally substituted by one or more halogen atoms, C₃₋₆-cycloalkyl, phenyl, COC₁₋₆alkyl, C₁₋₆alkoxy, OCF₃, hydroxy, hydroxy-C₁₋₆alkyl, hydroxy-C₁₋₆alkoxy, C₁₋₆alkoxy- C₁₋₆alkoxy, nitro, amino, C₁₋₆alkylamino, or di-C₁₋₆alkylamino;
n is 0, 1, 2, 3, 4 or 5;
R² is hydrogen, C₁₋₆alkyl or together with a group R³ forms a group —(CR⁶R⁷)ₚ— where R⁶ and R⁷ are independently hydrogen or C₁₋₆alkyl and p is 2, 3 or 4;
each R³ is independently C₁₋₆alkyl optionally substituted by one or more halogen atoms, halogen, C₁₋₆ alkoxy or together with the group R² forms a group —(CR⁶R⁷)ₚ— as defined above;
m is 0, 1 or 2;
each R⁴ is independently C₁₋₆ alkyl, or a group —X—R⁵ where X is a single bond, CH₂, O, NH or N—C₁₋₆alkyl;
k is 1 or 2;
R⁵ is an optionally substituted 5- to 7-membered heterocyclic ring or a bicyclic heterocyclic ring comprising 1 to 3 heteroatoms selected from nitrogen, sulfur or oxygen; and
Q is a phenyl ring or is a 6-membered heteroaryl ring comprising one or two nitrogen atoms.

2. The method of claim 1 wherein P is phenyl, naphthyl, benzofuryl or benzothienyl.

3. The method of claim 1 wherein R¹ is halogen, or a C₁₋₆alkyl group optionally substituted by one or more halogen atoms.

4. The method of claim 1 wherein R⁴ is a piperazine ring optionally substituted by C₁₋₆ alkyl.

5. The method of claim 1 wherein Q together with the phenyl group to which it is fused forms a quinoline, isoquinoline or quinazoline ring.

6. The method of claim 1 wherein R⁴ is a piperazine ring optionally substituted by C₁₋₆ alkyl; and Q together with the phenyl group to which it is fused forms a quinoline ring.

7. The method of claim 1 wherein the compound is selected from:
4-tert-butyl-N-(4-piperazin-1-yl-quinolin-6-yl)benzenesulfonamide or 5-chloro-3-methyl-benzo[b]thiophene-2-sulfonic acid (4-piperazin-1-yl-quinolin-6-yl) amide.

8. A method of treatment for the reduction of food intake, comprising administering to a patient in need of such treatment an effective amount of a compound, or a pharmaceutically acceptable salt or prodrug thereof, having a structure in accordance with Formula I: 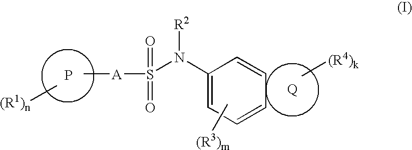
wherein
P is phenyl, naphthyl, a 5 or 6 membered heteroaryl ring comprising 1 to 3 heteroatoms selected from oxygen, nitrogen and sulfur, or a bicyclic or tricyclic heteroaryl ring comprising 1 to 3 heteroatoms selected from oxygen, nitrogen and sulfur;
A is a single bond, a C₁₋₆alkylene or a C₂₋₆alkenylene group;
each R¹ is independently halogen, C₁₋₆alkyl optionally substituted by one or more halogen atoms, C₃₋₆-cycloalkyl, phenyl, COC₁₋₆alkyl, C₁₋₆alkoxy, OCF₃, hydroxy, hydroxy-C₁₋₆alkyl, hydroxy-C₁₋₆alkoxy, C₁₋₆alkoxy-C₁₋₆alkoxy, nitro, amino, C₁₋₆alkylamino, or di-C₁₋₆alkylamino;
n is 0, 1, 2, 3, 4 or 5;
R² is hydrogen, C₁₋₆alkyl or together with a group R³ forms a group —(CR⁶R⁷)ₚ— where R⁶ and R⁷ are independently hydrogen or C₁₋₆alkyl and p is 2, 3 or 4;
each R³ is independently C₁₋₆alkyl optionally substituted by one or more halogen atoms, halogen, C₁₋₆ alkoxy or together with the group R² forms a group —(CR⁶R⁷)ₚ— as defined above;
m is 0, 1 or 2;
each R⁴ is independently C₁₋₆ alkyl, or a group —X—R⁵ where X is a single bond, CH₂, O, NH or N—C₁₋₆alkyl;
k is 1 or 2;
R⁵ is an optionally substituted 5- to 7-membered heterocyclic ring or a bicyclic heterocyclic ring comprising 1 to 3 heteroatoms selected from nitrogen, sulfur or oxygen; and
Q is a phenyl ring or is a 6-membered heteroaryl ring comprising one or two nitrogen atoms.

9. The method of claim 8 wherein P is phenyl, naphthyl, benzofuryl or benzothienyl.

10. The method of claim 8 wherein R¹ is halogen, or a C₁₋₆alkyl group optionally substituted by one or more halogen atoms.

11. The method of claim 8 wherein R⁴ is a piperazine ring optionally substituted by C₁₋₆ alkyl.

12. The method of claim 8 wherein Q together with the phenyl group to which it is fused forms a quinoline, isoquinoline or quinazoline ring.

13. The method of claim 8 wherein R⁴ is a piperazine ring optionally substituted by C₁₋₆ alkyl; and Q together with the phenyl group to which it is fused forms a quinoline ring.

14. The method of claim 8 wherein the compound is selected from:
4-tert-butyl-N-(4-piperazin-1-yl-quinolin-6-yl)benzenesulfonamide or 5-chloro-3-methyl-benzo[b]thiophene-2-sulfonic acid (4-piperazin-1-yl-quinolin-6-yl) amide.
